(19) United States
(12) Patent Application Publication
(10) Pub. No.: US 2023/0184729 A1
(43) Pub. Date: Jun. 15, 2023

(54) CALIBRATING OR VERIFYING AN OPERATION OF A VOLATILE ORGANIC COMPOUND SENSOR USING CARBON MONOXIDE

(71) Applicant: Carrier Corporation, Palm Beach Gardens, FL (US)

(72) Inventors: Callum Bailey, Lakewood Ranch, FL (US); Vipul H. Patel, Sarasota, FL (US)

(21) Appl. No.: 18/078,610

(22) Filed: Dec. 9, 2022

Related U.S. Application Data
(60) Provisional application No. 63/288,132, filed on Dec. 10, 2021.

(51) Int. Cl. G01N 33/00; G01N 27/416

(52) U.S. Cl. CPC G01N 33/0006; G01N 27/4163; G01N 33/0031

(57) ABSTRACT

A method of calibrating a detector that includes a volatile organic compound (VOC) sensor is provided. The method includes assembling the detector with the VOC sensor for field deployment and calibrating the VOC sensor using carbon monoxide (CO) as a calibrant prior to the field deployment.

### CROSS-REFERENCE TO RELATED APPLICATIONS

This application claims priority to U.S. Provisional Application Ser. No. 63/288,132, filed Dec. 10, 2021, the contents of which are hereby incorporated by reference in its entirety.

### BACKGROUND

The present disclosure relates to gas detectors and, more particularly, to mass and force indicators in diagnostics and output correction for metal oxide and electrochemical gas sensors in gas detectors.

Air pollution is the presence of substances in the atmosphere that are harmful to the health of humans and other living beings, or cause damage to the climate or to materials. There are many different types of air pollutants, such as gases (including ammonia, carbon monoxide, sulfur dioxide, nitrous oxides, methane, carbon dioxide and chlorofluorocarbons), particulates (both organic and inorganic) and biological molecules. These pollutants may be both inside (e.g., in residential and commercial buildings) and outside. Volatile organic compounds (VOCs) are a common indoor air pollutant, and have been linked to adverse health effects. As such, monitoring their levels with VOC sensors forms an important component of indoor air quality (IAQ) monitoring for ensuring a healthy building and a healthy living or work environment. A number of technologies have been developed to monitor VOCs, including metal oxide and electrochemical sensors. However to ensure accurate monitoring, the sensor operation may need to be verified both at the end of manufacturing and in the field (i.e., field calibration).

### BRIEF DESCRIPTION

According to an aspect of the disclosure, a method of calibrating a detector that includes a volatile organic compound (VOC) sensor is provided. The method includes assembling the detector with the VOC sensor for field deployment and calibrating the VOC sensor using carbon monoxide (CO) as a calibrant prior to the field deployment.

In accordance with additional or alternative embodiments, the calibrating of the VOC sensor includes exposing the VOC sensor to the CO, the VOC sensor includes at least one of a metal oxide sensor and an electrochemical sensor and the exposing of the VOC sensor to the CO includes positioning a sensor calibration tool close to the VOC sensor during the calibrating of the VOC sensor.

In accordance with additional or alternative embodiments, the detector includes the VOC sensor and a CO sensor, the calibrating of the VOC sensor includes exposing the VOC sensor to the CO and the exposing of the VOC sensor to the CO includes positioning a sensor calibration tool close to the VOC sensor and the CO sensor during the calibrating of the VOC sensor and a simultaneous calibrating of the CO sensor.

In accordance with additional or alternative embodiments, the detector includes the VOC sensor and a CO sensor, which is previously calibrated, the calibrating of the VOC sensor includes exposing the VOC sensor to the CO, the exposing of the VOC sensor to the CO includes positioning a sensor calibration tool close to the VOC sensor and the previously calibrated CO sensor during exposure of an unknown quantity of CO to the VOC sensor and the previously calibrated CO sensor and the previously calibrated CO sensor is used to measure a quantity of the CO by which the VOC sensor is calibrated.

According to an aspect of the disclosure, a method of calibrating a detector including a volatile organic compound (VOC) sensor and a carbon monoxide (CO) sensor is provided. The method includes assembling the detector with the VOC sensor for field deployment and calibrating the VOC sensor using CO as a calibrant prior to the field deployment.

In accordance with additional or alternative embodiments, the assembling of the VOC sensor is executed and completed in a manufacturing facility.

In accordance with additional or alternative embodiments, the calibrating of the VOC sensor includes exposing the VOC sensor to the CO, the VOC sensor includes at least one of a metal oxide sensor and an electrochemical sensor and the exposing of the VOC sensor to the CO includes positioning a sensor calibration tool close to the VOC sensor during the calibrating of the VOC sensor.

In accordance with additional or alternative embodiments, the calibrating of the VOC sensor includes exposing the VOC sensor to the CO and the exposing of the VOC sensor to the CO includes positioning a sensor calibration tool close to the VOC sensor and the CO sensor of the detector during the calibrating of the VOC sensor and a simultaneous calibrating of the CO sensor.

In accordance with additional or alternative embodiments, the calibrating of the VOC sensor includes exposing the VOC sensor to the CO, the CO sensor is previously calibrated, the exposing of the VOC sensor to the CO includes positioning a sensor calibration tool close to the VOC sensor and the previously calibrated CO sensor during exposure of an unknown quantity of CO to the VOC sensor and the previously calibrated CO sensor and the previously calibrated CO sensor is used to measure a quantity of the CO by which the VOC sensor is calibrated.

In accordance with additional or alternative embodiments, the CO sensor is configured to determine a verified amount of CO the detector is exposed to, the verified amount being used to calibrate the VOC sensor.

In accordance with additional or alternative embodiments, the calibrating of the VOC sensor includes exposing the VOC sensor to the CO for at least a relatively short exposure time of about 4-15 minutes.

In accordance with additional or alternative embodiments, the CO has about a 400 PPM concentration during the relatively short exposure time.

In accordance with additional or alternative embodiments, the method further includes re-calibrating the VOC sensor using the CO as the calibrant following the field deployment.

In accordance with additional or alternative embodiments, the re-calibrating is periodic, regularly scheduled or scheduled in response to a malfunction.

According to another aspect of the disclosure, a method of calibrating a detector including a volatile organic compound (VOC) sensor and a carbon monoxide (CO) sensor is provided. The method includes exposing the VOC sensor and the CO sensor to carbon monoxide (CO) as a calibrant and determining, using the CO sensor, a verified amount of CO the detector is exposed to, the verified amount being used to calibrate the VOC sensor.

In accordance with additional or alternative embodiments, the VOC sensor includes at least one of a metal oxide sensor and an electrochemical sensor and the exposing of the VOC sensor to the CO includes positioning a sensor calibration tool close to the VOC sensor during the calibrating of the VOC sensor.

In accordance with additional or alternative embodiments, the exposing of the VOC sensor to the CO includes positioning a sensor calibration tool close to the VOC sensor and the CO sensor of the detector during the calibrating of the VOC sensor and a simultaneous calibrating of the CO sensor.

In accordance with additional or alternative embodiments, the exposing of the VOC sensor to the CO includes positioning a sensor calibration tool close to the VOC sensor and the CO sensor of the detector during exposure of an unknown quantity of CO to the VOC sensor and the CO sensor and the CO sensor is previously-calibrated and is used to measure the quantity of CO and to use the measured value to calibrate the VOC sensor.

In accordance with additional or alternative embodiments, calibrating and re-calibrating of the VOC sensor includes exposing the VOC sensor to the CO for at least a relatively short exposure time.

In accordance with additional or alternative embodiments, the CO has about a 400 PPM concentration during the relatively short exposure time.

Additional features and advantages are realized through the techniques of the present disclosure. Other embodiments and aspects of the disclosure are described in detail herein and are considered a part of the claimed technical concept. For a better understanding of the disclosure with the advantages and the features, refer to the description and to the drawings.

### BRIEF DESCRIPTION OF THE DRAWINGS

For a more complete understanding of this disclosure, reference is now made to the following brief description, taken in connection with the accompanying drawings and detailed description, wherein like reference numerals represent like parts:

FIG. 1 is a flow diagram illustrating an exemplary method of calibrating a detector that includes a volatile organic compound (VOC) sensor and a carbon monoxide (CO) sensor in accordance with embodiments;

FIG. 2 is a schematic illustration of a manufacturing facility and a field deployment of a VOC sensor in accordance with embodiments

FIG. 3 is a graphical depiction of an exemplary calibration sequence of a VOC sensor in accordance with embodiments; and

FIG. 4 is a flow diagram illustrating an exemplary method of calibrating a detector that includes a VOC sensor and a CO sensor in accordance with embodiments.

### DETAILED DESCRIPTION

Volatile organic compounds (VOCs) are a common air pollutant and monitoring their levels with VOC sensors forms an important component of indoor air quality (IAQ) monitoring for ensuring a healthy building and a healthy living or work environment. A number of technologies have been developed to monitor VOCs, including metal oxide and electrochemical sensors. In these cases, sensor operations need to be verified at the end of the manufacturing line. Additionally, sensors may need field calibration once they are deployed and then over time to monitor degradation.

As will be described below, carbon monoxide (CO) is used to verify operations of VOC sensors at the end of the production line. Metal oxide sensors, some electrochemical sensors and additional VOC sensing technologies may be sensitive to CO in addition to conventional calibrant VOC gases such as ethanol. VOC sensor functionality verification can be performed at the same time as CO calibration processes for combination systems featuring CO detectors or alarms that also include a VOC sensor, providing a manufacturing efficiency. Furthermore, periodic field calibrations of the VOC sensor can be performed using CO gas as the calibrant and may allow for CO and VOC combination alarm and sensor systems to be calibrated in one step (e.g., via a technician using a smoke detector test kit that emits a controlled amount of CO).

It should be appreciated that these common smoke detector test kits may not be as controlled as would otherwise be ideal (e.g., there may be a wide range of CO released). By dual calibrating the CO sensor and the VOC sensor more accurate calibration of the VOC sensor may be possible, even while using the non-precise smoke detector test kits. For example, the CO sensor (which is an extremely precise sensor) may be used to verify the amount of CO that the VOC sensor is being exposed to and thus enable a more precise calibration of the VOC sensor.

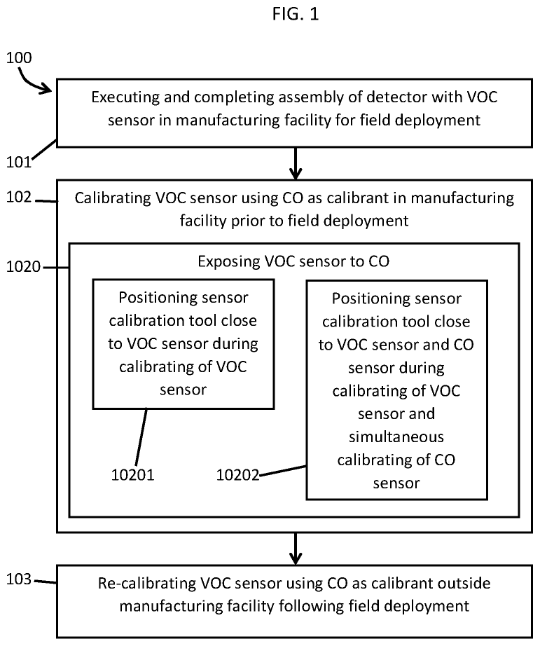

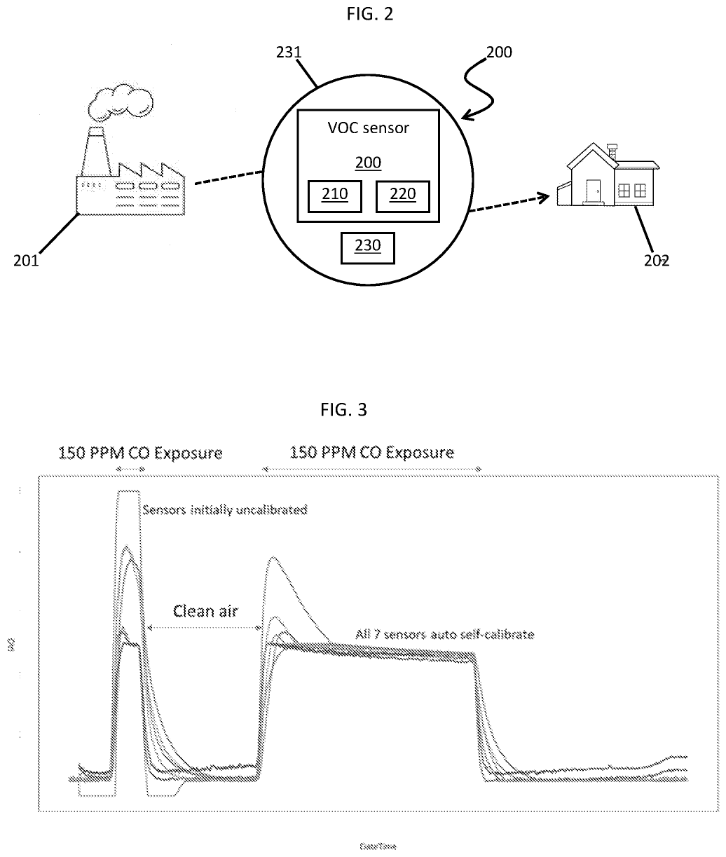

With reference to FIGS. 1 and 2, a method 100 of calibrating a detector 231 including a VOC sensor 200 and a CO sensor is provided.As shown in FIG. 1, the method 100 includes executing and completing an assembly of the VOC sensor 200 (see FIG. 2) in a manufacturing facility 201 (see FIG. 2) for field deployment (block 101) in a given space, such as the interior of a building or a house 202 (see FIG. 2). The method further includes calibrating the VOC sensor 200 using carbon monoxide (CO) as a calibrant in the manufacturing facility 201 prior to the field deployment (block 102) by exposing the VOC sensor 200 to the CO (block 1020). In addition, the method can include re-calibrating the VOC sensor 200 using the CO as the calibrant outside of the manufacturing facility 201 following the field deployment (block 103).

The VOC sensor 200 can include at least one of a metal oxide sensor 210 and an electrochemical sensor 220. In these or other cases, the exposing of the VOC sensor 200 to the CO of block 1020 can include positioning a sensor calibration tool close to the VOC sensor 200 during the calibrating of the VOC sensor 200 (block 10201). In some cases, the VOC sensor 200 can be paired with the CO sensor 230 in the detector 231. The CO sensor 230 can be configured to determine a verified amount of CO the single detector 231 is exposed to, where the verified amount can be used to calibrate the VOC sensor 200. In these or other cases, the exposing of the VOC sensor 200 to the CO of block 1020 can include positioning the sensor calibration tool close to the VOC sensor 200 and the CO sensor 230 of the detector 231 during the calibrating of the VOC sensor 200 and a simultaneous calibrating of the CO sensor 230 (block 10202).

In any case, with reference to FIG. 3, the calibrating of the VOC sensor of block 102 and the re-calibrating of the VOC sensor of block 103 can include exposing the VOC sensor 200 to the CO (having a concentration of about 400 PPM) for a relatively short exposure time of about 4-15 minutes. In some but not all cases, the calibrating of the VOC sensor of block 102 and the re-calibrating of the VOC sensor of block 103 can further include exposing the VOC sensor to the CO (having a concentration of about 150 PPM) for a relatively long exposure time following a delay after completion of the relatively short exposure time to confirm calibration status.

In accordance with embodiments, the re-calibrating of the VOC sensor 200 of block 103 can be periodic, regularly scheduled, or scheduled in response to a malfunction.

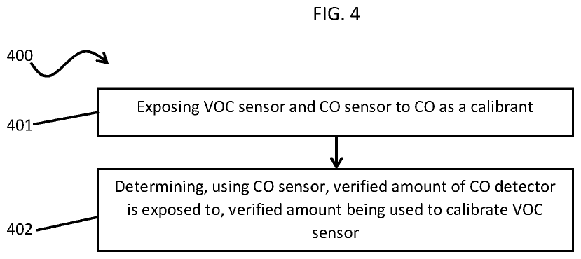

With reference to FIG. 4, a method 400 of calibrating a detector that includes a VOC sensor and a CO sensor, such as the detector 231 described above, is provided. As shown in FIG. 4, the method 400 includes exposing the VOC sensor and the CO sensor to CO as a calibrant (block 401) and determining, using the CO sensor, a verified amount of CO the detector is exposed to, the verified amount being used to calibrate the VOC sensor (block 402).

Technical effects and benefits of the present disclosure are the use of CO as a calibrant to thereby avoid the need for additional calibration systems to be installed in production of VOC systems in facilities that already perform CO calibration for CO alarms and detectors. Furthermore, systems that perform field calibration of CO can also be utilized to perform periodic field calibration of VOC sensors without the need for additional equipment and fulfil standards requirements for periodic calibration. Field calibration of VOC sensors can be offered as a service.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the technical concepts in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

While the preferred embodiments to the disclosure have been described, it will be understood that those skilled in the art, both now and in the future, may make various improvements and enhancements which fall within the scope of the claims which follow. These claims should be construed to maintain the proper protection for the disclosure first described.

What is claimed is:

1. A method of calibrating a detector comprising a volatile organic compound (VOC) sensor, the method comprising:
assembling the detector with the VOC sensor for field deployment; and
calibrating the VOC sensor using carbon monoxide (CO) as a calibrant prior to the field deployment.

2. The method according to claim 1, wherein:
the calibrating of the VOC sensor comprises exposing the VOC sensor to the CO,
the VOC sensor comprises at least one of a metal oxide sensor and an electrochemical sensor, and
the exposing of the VOC sensor to the CO comprises positioning a sensor calibration tool close to the VOC sensor during the calibrating of the VOC sensor.

3. The method according to claim 1, wherein:
the detector comprises the VOC sensor and a CO sensor,
the calibrating of the VOC sensor comprises exposing the VOC sensor to the CO, and
the exposing of the VOC sensor to the CO comprises positioning a sensor calibration tool close to the VOC sensor and the CO sensor during the calibrating of the VOC sensor and a simultaneous calibrating of the CO sensor.

4. The method according to claim 1, wherein:
the detector comprises the VOC sensor and a CO sensor, which is previously calibrated,
the calibrating of the VOC sensor comprises exposing the VOC sensor to the CO,
the exposing of the VOC sensor to the CO comprises positioning a sensor calibration tool close to the VOC sensor and the previously calibrated CO sensor during exposure of an unknown quantity of CO to the VOC sensor and the previously calibrated CO sensor, and
the previously calibrated CO sensor is used to measure a quantity of the CO by which the VOC sensor is calibrated

5. A method of calibrating a detector comprising a volatile organic compound (VOC) sensor and a carbon monoxide (CO) sensor, the method comprising:
assembling the detector with the VOC sensor for field deployment; and
calibrating the VOC sensor using CO as a calibrant prior to the field deployment.

6. The method according to claim 5, wherein the assembling of the VOC sensor is executed and completed in a manufacturing facility.

7. The method according to claim 5, wherein:
the calibrating of the VOC sensor comprises exposing the VOC sensor to the CO,
the VOC sensor comprises at least one of a metal oxide sensor and an electrochemical sensor, and
the exposing of the VOC sensor to the CO comprises positioning a sensor calibration tool close to the VOC sensor during the calibrating of the VOC sensor.

8. The method according to claim 5, wherein:
the calibrating of the VOC sensor comprises exposing the VOC sensor to the CO, and
the exposing of the VOC sensor to the CO comprises positioning a sensor calibration tool close to the VOC sensor and the CO sensor of the detector during the calibrating of the VOC sensor and a simultaneous calibrating of the CO sensor.

9. The method according to claim 5, wherein:
the calibrating of the VOC sensor comprises exposing the VOC sensor to the CO,
the CO sensor is previously calibrated,
the exposing of the VOC sensor to the CO comprises positioning a sensor calibration tool close to the VOC sensor and the previously calibrated CO sensor during exposure of an unknown quantity of CO to the VOC sensor and the previously calibrated CO sensor, and
the previously calibrated CO sensor is used to measure a quantity of the CO by which the VOC sensor is calibrated.

10. The method according to claim 5, wherein the CO sensor is configured to determine a verified amount of CO the detector is exposed to, the verified amount being used to calibrate the VOC sensor.

11. The method according to claim 5, wherein the calibrating of the VOC sensor comprises exposing the VOC sensor to the CO for at least a relatively short exposure time of about 4-15 minutes.

12. The method according to claim 11, wherein the CO has about a 400 PPM concentration during the relatively short exposure time.

13. The method according to claim 5, further comprising re-calibrating the VOC sensor using the CO as the calibrant following the field deployment.

14. The method according to claim 13, wherein the re-calibrating is periodic, regularly scheduled or scheduled in response to a malfunction.

15. A method of calibrating a detector comprising a volatile organic compound (VOC) sensor and a carbon monoxide (CO) sensor, the method comprising:
exposing the VOC sensor and the CO sensor to carbon monoxide (CO) as a calibrant; and
determining, using the CO sensor, a verified amount of CO the detector is exposed to, the verified amount being used to calibrate the VOC sensor.

16. The method according to claim 15, wherein:
the VOC sensor comprises at least one of a metal oxide sensor and an electrochemical sensor, and
the exposing of the VOC sensor to the CO comprises positioning a sensor calibration tool close to the VOC sensor during the calibrating of the VOC sensor.

17. The method according to claim 15, wherein the exposing of the VOC sensor to the CO comprises positioning a sensor calibration tool close to the VOC sensor and the CO sensor of the detector during the calibrating of the VOC sensor and a simultaneous calibrating of the CO sensor.

18. The method according to claim 15, wherein:
the exposing of the VOC sensor to the CO comprises positioning a sensor calibration tool close to the VOC sensor and the CO sensor of the detector during exposure of an unknown quantity of CO to the VOC sensor and the CO sensor, and
the CO sensor is previously-calibrated and is used to measure the quantity of CO and to use the measured value to calibrate the VOC sensor.

19. The method according to claim 15, wherein calibrating and re-calibrating of the VOC sensor comprises exposing the VOC sensor to the CO for at least a relatively short exposure time.

20. The method according to claim 19, wherein the CO has about a 400 PPM concentration during the relatively short exposure time.
